(19) United States
(12) Patent Application Publication
(10) Pub. No.: US 2024/0401159 A1
(43) Pub. Date: Dec. 5, 2024

(54) METHODS FOR PARALLEL LAMP ASSAYS AT A SINGLE TEMPERATURE USING TEMPERATURE-SHIFTING AGENTS

(71) Applicant: ARIZONA BOARD OF REGENTS ON BEHALF OF ARIZONA STATE UNIVERSITY, Scottsdale, AZ (US)

(72) Inventors: Karen ANDERSON, Scottsdale, AZ (US); Mark KNAPPENBERGER, Mesa, AZ (US); Siril AROCKIAM, Tempe, AZ (US)

(21) Appl. No.: 18/694,303

(22) PCT Filed: Sep. 22, 2022
(86) PCT No.: PCT/US2022/044352

§ 371 Date: Mar. 21, 2024

Related U.S. Application Data
(60) Provisional application No. 63/246,959, filed on Sep. 22, 2021.

(51) Int. Cl. C12Q 1/70; C12Q 1/6848

(52) U.S. Cl. CPC C12Q 1/701; C12Q 1/6848

(57) ABSTRACT

Provided herein are methods for multiplexed LAMP-based detection of multiple target nucleic acids in parallel at a single reaction temperature. In particular, provided herein are methods for performing a plurality of LAMP reactions in parallel using the addition of LAMP reaction temperature-shifting agents to better optimize a subset of the LAMP reactions to a single reaction temperature. For certain LAMP reactions, provided herein are LAMP primer sets and respective LAMP temperature-shifting parameters to provide efficient LAMP readouts at specific temperatures. Also provided herein are methods for increasing end-point signal-to-noise ratios of LAMP assays based on the detection of a fluorescent intercalating-dye in an amplification product.

### CROSS-REFERENCE TO RELATED APPLICATIONS

The present application claims priority to U.S. Provisional Patent Application No. 63/246,959, filed Sep. 22, 2021, the entire contents of which are hereby incorporated by reference.

### BACKGROUND

Nucleic acid detection methods are important tools for identifying diseases, managing infectious diseases, monitoring health conditions, and directing treatment decisions. PCR-based techniques have disadvantages, including the need for trained technicians and expensive equipment restricted to centralized laboratories. Alternative tools to PCR are loop mediated isothermal amplification (LAMP) based techniques. LAMP assays can be highly sensitive, rapid, and low cost. LAMP assays are often more than twice as fast as PCR assays, and in some cases, LAMP techniques are more sensitive than conventional PCR techniques. Furthermore, LAMP-based methods can provide direct visualization of results by the naked eye and/or in real time as reactions proceed. In some cases, LAMP reaction results can be detected and recorded using common items such as with a mobile phone camera, photocopy machine, or office scanner.

LAMP assays have been developed to detect clinically important target nucleic acids, including from SARS-CoV-2. However, LAMP approaches can be hindered by primer design, parameter optimization, and artefacts. For example, every LAMP reaction has a complex primer design of a LAMP primer set of four or six primers targeting six or eight regions within a relatively small target sequence where efficient target amplification will occur only within a narrow temperature range. This limits how many LAMP reactions can be performed simultaneously at a given temperature. There is a need in the art for improved methods of LAMP in order to cost-effectively detect multiple nucleic acids in samples using multiple primer sets for various amplifications, including to detect multiple viral target sequences of SARS-CoV-2 or other viruses. In particular, there is a need for LAMP methods that allow for low-cost detection of multiple nucleic acid targets within a single sample in parallel all performed at a single reaction temperature. There is also a need for LAMP methods that produce more reliable and accurate results by providing more optimal signal-to-noise ratios of LAMP reaction end products.

### SUMMARY

The present disclosure provides methods of detecting nucleic acids of interest for multiple LAMP reactions in parallel at a single reaction temperature despite the fact that some of the LAMP reactions are conventionally optimized a different temperature. This is accomplished by using a reaction temperature-shifting agent(s) to re-optimize the reaction temperature of a subset of the LAMP reactions performed in parallel.

In a first aspect, provided herein is a method of performing a plurality of loop-mediated isothermal amplification (LAMP) assays in parallel at a single temperature. The method can comprise (a) providing a plurality of LAMP reactions, wherein each LAMP reaction comprises a LAMP primer set, a sample comprising nucleic acids, and a temperature-resistant strand displacing DNA polymerase and wherein at least one LAMP reaction comprises at least one LAMP-temperature shifting agent allowing for more efficient amplification at a set temperature compared to the same LAMP reaction in the absence of the at least one LAMP-temperature shifting agent; (b) performing a plurality of LAMP reactions at the set temperature, wherein at least two of the LAMP reactions comprise different LAMP primer sets; and (c) detecting LAMP reaction products produced by the plurality of LAMP reactions.

In a further aspect, provided herein is a method for increasing an end-point signal-to-noise ratio of a fluorescent intercalating-dye based LAMP assay, the method comprising: (a) performing one or more LAMP reactions with a fluorescent intercalating-dye; (b) heating the one or more LAMP reactions at temperature of at least about 80° C.; and (c) detecting a LAMP reaction product by detecting the fluorescent intercalating-dye.

Also provided is a kit comprising a LAMP primer described herein and at least one LAMP-temperature shifting agent.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A-1B shows the effect of different LAMP reaction additives on LAMP performance. FIG. 1A shows the effect of reaction additives on mean time to detectable amplification (Ct) for 1000 copies of SARS-CoV-2 with nucleocapsid-specific primers. The mean time to amplification of LAMP amplification products for three independent reactions is graphed for the additives: 40 mM Gu-HCl, 40 μg glycogen, 2% DMSO, 2% ethanol (EtOH), and 1% glycerol. FIG. 1B shows the effect of different LAMP reaction additives on LAMP amplicon melting temperature for the N2_001 primer set. The mean melting temperature for three independent reactions is graphed for the additives: 40 mM Gu-HCl, 40 μg glycogen, 2% DMSO, 2% ethanol (EtOH), and 1% glycerol. The Gu-HCl additive was capable of increasing the melting temperature whereas the additives DMSO and ethanol were capable of decreasing the melting temperature of the amplicon.

FIG. 2 shows the signal to noise ratio as a function of measurement temperature for a fluorescence-based LAMP reaction. Above about 85° C., desired LAMP amplification products (signal) can be detected with less noise from undesirable signals, e.g. caused by the presence of non-specific products and primer dimers.

FIG. 3 shows the derivative reporter of fluorescent dye dissociation graphed over temperature. Above about 85° C., the fluorescent dye begins dissociating from LAMP amplification products.

### DETAILED DESCRIPTION

The methods and compositions provided herein are based at least in part on the inventors' development of methods of optimizing LAMP reactions to new reaction temperatures using temperature-shifting additives to achieve efficient amplification and detection. These methods were applied to multiple LAMP reactions to achieve efficient amplification and detection at a single chosen temperature. The re-optimization of LAMP reactions to a given temperature allows these reactions to be run simultaneously along with other LAMP reactions conventionally optimized to the same temperature without any additive. As described herein, this development provides for multiplexing a plurality of LAMP reactions in parallel at a single set temperature, which would fail in the absence of the addition of an effective amount of a temperature-shifting agent to the re-optimized LAMP reactions. Furthermore, the inventors demonstrate how to generally optimize LAMP reactions to new temperatures using the addition of LAMP temperature-shifting additives as well as optimizing other parameters including, for example, primer design and melting energy input.

In a first aspect, the disclosure provides methods of performing a plurality of LAMP reactions in parallel at a single temperature wherein at least one LAMP reaction containing a LAMP-temperature shifting agent allows for more efficient product amplification than the same LAMP reaction performed the same way except without the LAMP-temperature shifting agent. In another aspect, provided herein are methods for increasing end-point signal-to-noise ratios for a fluorescent intercalating-dye based LAMP assay by denaturing undesirable double-stranded (ds) DNAs while preserving dsDNA pairing of desired LAMP reaction products using a heating step after the LAMP reaction(s) are completed in order to reduce the occurrence and magnitude of artifactual signals.

The methods herein can comprise one or more LAMP reactions containing a sample comprising nucleic acids. The sample comprising nucleic acids may be a biological sample or non-biological sample. Non-limiting examples of samples include diagnostic tissue samples, environmental samples, food or water samples, agricultural products, and nucleic acid extracts of any of the aforementioned. A sample comprising one or more nucleic acids may or may not comprise a target nucleic acid of interest.

The methods of this disclosure can be performed using a sample of or from any biological entity, including without limitation one or more organisms, humans, non-human mammals, cells, bacteria, fungi, algae, or viruses. The term “sample” includes all types of tissue samples taken from a subject, such as, e.g., animal or plant tissue, buccal smears, samples obtained from biopsy or bodily fluids (e.g., blood, mucus, saliva, sputum, urine, amniotic fluid, etc.).

Samples are prepared for use as nucleic acid templates in the methods of this disclosure using conventional techniques, for example, a nucleic acid extraction. A sample comprising nucleic acids may be prepared from samples using conventional techniques, which typically depend on the source from which a sample is taken, including but not limited to a simple nucleic acid extraction comprising a cell lysis step, a centrifugation step to remove cellular debris, a proteolytic step to degrade proteins, an organic solvent precipitation step to remove proteins (e.g. using phenol or a mixture of phenol and chloroform), and a nucleic acid precipitation step using an alcohol (e.g. ethanol or isopropanol). Alternative nucleic acid sample preparation methods known in the art include silica-based methods for nucleic acid adsorption (e.g. silica membranes, beads or particles), magnetic-based solid support technologies (e.g. magnetic beads), anion exchange methods, and chromatographic methods using different resins. In some embodiments, the nucleic acid template sample is first prepared using a reverse transcriptase reaction known in the art. In some cases, the sample comprising nucleic acids undergoes no preparation step, i.e. a raw sample. In some cases, samples are heated prior to performing the other method steps. For instance, samples can be heated at a temperature of about 65° C. or greater, which kills the virus and releases nucleic acids. In other cases, samples are frozen (e.g., at −80° C.) prior to performing the other method steps.

The methods of this disclosure can be performed using a sample which is non-biological either in whole or in part. Non-biological samples include, without limitation, clothing fibers, paper, plastic and packaging materials, swabs of objects and surfaces, and nucleic acid extracts of any of the aforementioned. In some cases, samples are obtained by swabbing, washing, or otherwise collecting biological material from a non-biological object such as a medical device, medical instrument, handrail, doorknob, etc.

In some embodiments, the sample is a biological sample obtained from an individual subject (e.g., a human subject, a non-human mammalian subject) or a collection of subjects. A subject of the current disclosure includes a prokaryote, eukaryote, alga, fungus, plant, virus, and species of Metazoa. The term “subject” may be used interchangeably with the terms “individual” and “patient” and includes human and non-human subjects. In some embodiments, the subject is a mammal, such as a domesticated or pet mammal.

The sample is, in some embodiments, a diagnostic sample. The sample type will vary depending on the target nucleic acid of interest of the LAMP assay. The biological sample can be saliva, a nasopharyngeal swab, blood, serum, sputum, or another matrix. For example, a diagnostic sample for detecting viruses such as SARS-CoV-2 can be a saliva sample, a mucus sample, a nasopharyngeal swab sample, a sputum sample, or a blood sample.

In some embodiments, the plurality of LAMP reactions performed in parallel each comprises an equivalent sample for the sample comprising nucleic acids. As used herein, “equivalent” samples are samples from the same source or having the same origin and utilized at the same concentration or quantity in the LAMP reaction. A non-limiting example of equivalent samples are equal-sized aliquots taken from a single sample composition.

As used herein, a “LAMP reaction” refers to a loop mediated isothermal amplification (LAMP) reaction. Typically, a LAMP reaction takes about 20 minutes to about one hour and is performed at a set temperature between 60-85° C. to optimize template melting and primer annealing. Typically, a LAMP reaction comprises (1) a thermostable strand displacing polymerase, (2) a sample comprising nucleic acid (e.g. a RNA or DNA nucleic acid template), (3) at least four or more sequence specific primers, (4) nucleotide triphosphates, (5) a divalent metal ion (e.g. Mg⁺²), and (6) a buffer. Some LAMP reactions further comprise a detectable agent, such as, e.g., a dye or nucleic acid probe.

As used herein, a “LAMP assay” refers to performing at least one LAMP reaction in order to detect the presence or absence of a target nucleic acid sequence or sequences.

The term “LAMP primer set” refers to a set of four, five, six or more LAMP primers which is capable (under optimized conditions) of producing a LAMP reaction product when a target nucleic acid template is present in the reaction (e.g. temperature and template concentration). In some embodiments, a LAMP primer set comprises two inner primers (FIP and BIP) and two outer primers (F3 and B3). In other embodiments, a LAMP primer set comprises two inner primers (FIP and BIP), two outer primers (F3 and B3), and two loop primers (LF and LB).

As used herein, the terms “nucleic acid of interest,” and “target nucleic acid” include a nucleic acid originating from one or more biological entities within a sample. The target nucleic acid of interest to be detected in a sample can be a sequence or a subsequence from DNA, such as nuclear or mitochondrial DNA, or cDNA that is reverse transcribed from RNA in the sample. The sequence of interest can also be from RNA, such as mRNA, rRNA, tRNA, miRNA, siRNAs, antisense RNAs, or long noncoding RNAs. More generally, the sequences of interest can be selected from any combination of sequences or subsequences in the genome or transcriptome of a species or an environment. In some cases, a defined set of primers are designed to amplify in a LAMP reaction target nucleic acids that would be expected in a sample, for example a gene or mRNA.

The term “temperature-resistant strand displacing DNA polymerase” refers to any polymerase capable of isothermal LAMP amplification over a range of temperatures including over 50, 60, 70, 80° C. or greater. Such DNA polymerases are capable of displacing one strand of a double-stranded nucleic acid template during DNA synthesis. Non-limiting examples of temperature-resistant strand displacing DNA polymerases are Bst and thermostable reverse transcriptases like RTx. The term “Bst” refers to an enzymatic portion of a family of Bacillus DNA polymerases known in the art, such as, e.g., from B. stearothermophilus and other closely related moderately thermophilic species. The term “RTx” refers to a thermostable reverse transcriptase known in the art.

As used herein, the term “divalent metal ion” refers to an ion having a 2+ charge. Non-limiting examples of divalent metal ions include barium, beryllium, cadmium, calcium, chromium (II), cobalt (II), copper (II), iron (II), lead (II), magnesium, manganese (II), nickel (II), strontium, zinc, and tin (II). In some embodiments, the divalent metal ion serves as a cofactor to a DNA polymerase. In some embodiments, the divalent metal ion is selected from Mg²⁺, Mn²⁺, Co²⁺, Ni²⁺, Zn²⁺, Cu²⁺, and Cd²⁺. In some embodiments, the divalent metal ion is selected from Mg²⁺, Mn²⁺, Co²⁺, and Cd²⁺. In some embodiments, the divalent metal ion is selected from Mg²⁺and Mn²⁺.

The disclosure provides methods of performing a plurality of LAMP reactions in parallel at a single temperature wherein at least one LAMP reaction comprises at least one LAMP-temperature shifting agent allowing for more efficient amplification at a set temperature compared to the same LAMP reaction in the absence of the at least one LAMP-temperature shifting agent. The term “LAMP-temperature shifting agent” as used herein refers to an agent not typically considered useful to include in a LAMP reaction but capable when present in a LAMP reaction of altering the optimal temperature for LAMP amplification away from the predicted ideal LAMP reaction temperature for the LAMP reaction under ideal conditions and in the absence of the agent. Non-limiting examples of LAMP temperature shift agents include dimethyl sulfoxide (DMSO), ethanol, glycerol, glycogen, and guanidine hydrochloride (Gu-HCl).

The optimal temperature for a LAMP reaction comprising a specific amount or concentration of at least one LAMP-temperature shifting agent can differ from the same LAMP reaction performed without the LAMP-temperature shifting agent. Further, the amount or concentration of the LAMP-temperature shifting agent can be chosen to shift the optimal temperature of a given LAMP reaction closer to a desired set temperature which differs from the predicted ideal LAMP reaction temperature for the same reaction performed under ideal conditions (e.g. the predicted ideal temperature for the same LAMP reaction unadulterated by the addition of the at least one LAMP-temperature shifting agent).

The term “additive” as used herein refers to a compound or agent not typically considered useful to include in a LAMP reaction, such as a LAMP-temperature shifting agent described above.

In some embodiments, the method comprises (a) providing a plurality of LAMP reactions, wherein each LAMP reaction comprises a LAMP primer set, a sample comprising nucleic acids, and a temperature-resistant strand displacing DNA polymerase and wherein at least one LAMP reaction comprises at least one LAMP-temperature shifting agent allowing for more efficient amplification at a set temperature compared to the same LAMP reaction in the absence of the at least one LAMP-temperature shifting agent; (b) performing a plurality of LAMP reactions at the set temperature, wherein at least two of the LAMP reactions comprise different LAMP primer sets; and (c) detecting LAMP reaction products produced by the plurality of LAMP reactions. In some embodiments, the set temperature of the plurality of LAMP reactions step (b) is controlled by a water bath, heating element, and/or thermal cycler. In some embodiments, the plurality of LAMP reactions are each physically separated from each other within a multi-well plate, plurality of tubes, or plurality of cells, chambers, or spots of a chip or slide.

The term “detecting,” “detect” or “detection” as used herein indicates the determination of the existence or presence of a target molecule or signal in a limited portion of space, including but not limited to a sample, a reaction mixture, a molecular complex and a substrate including a platform and an array. Detection is “quantitative” when it refers, relates to, or involves the measurement of quantity or amount of the target molecule or signal (also referred to as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through a detectable light signal(s): fluorescence, spectra, or images of a LAMP reaction of interest obtained during a detecting step.

As used herein, the term “detectable” means the molecule or atom can be detected by a known process, whether directly or indirectly, such as, after a processing step, chemical reaction, enzymatic reaction, and/or the addition of an additional reagent(s). For example, a “detectable agent” is an agent which can be detected by a known process, whether directly or indirectly. Non-limiting examples of detectable agents include fluorophores, colorimetric agents, dyes, biochemical affinity tags, and bioluminescent agents.

In the methods of the disclosure, a LAMP reaction product may be detected by any means known in the art and/or described herein. Non-limiting examples of detecting include using a fluorescent agent, colorimetric agent, a labeled nucleic acid hybridization probe. Non-limiting examples of detecting include photometric methods of detecting light absorbance, absorption (e.g. color), emission upon excitation (e.g. fluorescence), and/or scattering (e.g. caused by magnesium pyrophosphate precipitation). In some embodiments, the detecting comprises detecting a change, for e.g., in color, pH, pyrophosphate levels, fluorescence, or turbidity, as compared to the starting reaction. In some embodiments, the detecting step comprises adding a detectable agent to each LAMP reaction, such as before or after the performing the step.

In some cases, a LAMP reaction product is detected by changes in color (e.g. caused by a pH sensitive colorimetric agent), fluorescence (e.g. caused by a dsDNA intercalating dye), or turbidity (e.g. caused by magnesium pyrophosphate precipitation). In some embodiments, two or more colorimetric agents (e.g. pH sensitive colorimetric agents) are used together in a single LAMP reaction for detecting LAMP reaction product(s). Certain color and/or fluorescence changes indicating the presence of a LAMP reaction product may be detected visually by the naked eye. Non-limiting examples of bioluminescent detection include using BART and ELIDA, which can follow dynamic changes in pyrophosphate levels during a LAMP reaction.

Non-limiting examples of detecting include monitoring fluorescence of a LAMP reaction, such as, e.g., changes in emission spectra at a given excitation wavelength, which may be caused by, e.g. frequency resonance energy transfer (FRET) or target binding by a molecular beacon. Non-limiting examples of detectable fluorophores include fluorescein dyes (e.g. calcein), cyanine dyes, acridine orange, ethidium bromide, methylene blue, propidium iodide, and propidium monoazide. In some embodiments, the detectable agent is a cyanine dye which intercalates into dsDNA. In some embodiments, the divalent metal ion is manganese and the detectable agent (e.g. calcein) fluoresces as free manganese levels increase.

Non-limiting examples of detecting include monitoring LAMP reaction color using a colorimetric agent. Non-limiting examples of detectable colorimetric agents include agents which change color based on the presence or absence of alkaline earth metals ions, such as, e.g., hydroxyl napthol blue (HNB) or based on the pH. As used herein, the term “pH sensitive colorimetric agent” refers to an agent which enables colorimetric detection of a pH change. Non-limiting examples of pH sensitive colorimetric agents include phenol red, bromophenol blue, bromothymol blue, bromocresol purple, cresol red, neutral red, phthalein dyes (e.g. o-cresolphthalein, α-naphtholphthalein, and phenolphthalein) and pH-sensitive indicator dyes, such as, e.g., a fluorescein dye, azaBODIPY dye, acetoxymethyl ester (BCECF-AM), and 5-(and 6)-carboxy SNARF-1.

In some embodiments, LAMP reaction products are detected with a labeled nucleic acid probe designed to hybridize to a LAMP reaction product. In some embodiments, a LAMP reaction product is detected with a labeled hybridization probe comprising gold (Au) atoms which is capable of a color change or preventing a color change caused by the aggregation of unbound probe. The terms “hybridize” and “hybridization” as used herein refer to the association of two nucleic acids to form a stable duplex. Nucleic acids hybridize due to a variety of well characterized physico-chemical forces, such as hydrogen bonding, solvent exclusion, base stacking and the like. An extensive guide to the hybridization of nucleic acids is found in Tijssen, P., editor. 1993. Laboratory Techniques in Biochemistry and Molecular Biology: Hybridization with Nucleic Acid Probes (Vol. 24, Part II, Elsevier B.V., N.Y.). One of skill in the art will understand that “hybridization” as used herein does not require a precise base-for-base complementarity. That is, a duplex can form, between two nucleic acids that contain mismatched base pairs. The conditions under which nucleic acids that are perfectly complementary or that contain mismatched base pairs will hybridize to form a duplex are well known in the art and are described, for example, in Sambrook, J. and Russell, D., editors. 2001. Molecular Cloning: A Laboratory Manual (3^{rd}ed., Cold Spring Harbor Laboratory Press, NY).

As used herein, the term “complementary” refers to a nucleic acid that forms a stable duplex with its “complement”. For example, nucleotide sequences that are complementary to each other have mismatches at less than 20% of the bases, at less than about 10% of the bases, at less than about 5% of the bases, and more preferably have no mismatches.

For some embodiments, the detecting comprises displaying an indication of detecting results on a device, such as using an analog light or digital display. In some embodiments, the LAMP primers are labeled, such as, e.g., with a fluorophore or biotin tag. In some embodiments, LAMP reaction products are detected by an electrophoretic and/or lateral flow assay. In some embodiments, the LAMP reaction is treated with an RNase, such as, e.g. an RNase H. In some embodiments, a LAMP reaction product is treated with a restriction enzyme.

In some embodiments, the LAMP reactions are performed in the presence of a buffer with less robust or has minimal buffering capacity (e.g. a weakly buffered reaction) such that as the LAMP reaction proceeds, the pH changes more quickly than in the presence of a more robustly buffered LAMP reaction. In some embodiments, when a LAMP reaction changes from red to yellow, this indicates the presence of a LAMP reaction product.

The term “ambient temperature” refers to a temperature approximately equivalent to the air temperature of a LAMP device or the environment in which a LAMP reaction or LAMP device is present without the addition of heat, such as, e.g. by the activation of a heating element or thermocycler thereby increasing the temperature.

In some embodiments, the temperature-resistant strand displacing DNA polymerase is inactive at temperatures of about 45, 40, 35, or 30° C. because of the presence of an inhibitory aptamer which binds the polymerase but wherein the aptamer loses inhibitory activity at temperatures over 35, 40, 45, or 50° C.

In some embodiments, the LAMP reaction is incubated at ambient temperature prior to the performing step of the method. In some embodiments, the incubating is for at least 5, 10, 20, or 30 minutes.

In some embodiments, carryover contamination is minimized by the use of deoxyuridine triphosphate (dUTP) instead of deoxythymidine triphosphate (dTTP) in the deoxyribonucleotides of one or more LAMP reactions combined with the addition of uracil DNA glycosylase (UDG) to the LAMP reaction.

In some embodiments, the LAMP-temperature shifting agent is DMSO and is present in a LAMP reaction at about 1-5% volume per volume (v/v). In some further embodiments, the presence of the DMSO decreases the melting temperature of the primary amplifaction product in the LAMP reaction. The temperature may be decreased by at least 1, 2, 3, or 4 degrees C.

In some embodiments, the LAMP-temperature shifting agent is ethanol and is present in a LAMP reaction at about 1.5-3% (v/v). In some further embodiments, the presence of the ethanol decreases the melting temperature of the primary amplifaction product in the LAMP reaction. The temperature may be decreased by at least 1, 2, 3, or 4 degrees C.

In some embodiments, the LAMP-temperature shifting agent is glycerol and is present in a LAMP reaction at about 0.5-5% (v/v). In some further embodiments, the presence of the DMSO decreases the melting temperature of the primary amplifaction product in the LAMP reaction. The temperature may be decreased by at least 1, 2, 3, or 4 degrees C.

In some embodiments, the LAMP-temperature shifting agent is glycogen and is present in a LAMP reaction at about 1-4 micrograms (μg) per microliter (μL) or about 20-80 μg per 20 μL. In some further embodiments, the glycogen alters the melting temperature of the primary amplifaction product in the LAMP reaction. The temperature may be altered by at least 1, 2, or 3 degrees C.

In some embodiments, the LAMP-temperature shifting agent is Gu-HCl and is present in a LAMP reaction at about 5 to 100 millimolar (mM). In some further embodiments, the Gu-HCl increases the melting temperature of the primary amplifaction product in the LAMP reaction. The temperature may be increased by at least 1, 2, 3, or 4 degrees C.

The skilled worker will be able to alter the design of a given LAMP assay to produce more clear detection results, such as, e.g. by changing the detecting step, detectable agent, the timing of detecting, photometric setup, optical parameters, etc.

Provided herein are methods of performing LAMP assays for amplification of specific target nucleic acids, such as, e.g. to detect genetic sequences from viruses and humans. In some embodiments, the viral target nucleic acid is a SARS-CoV-2 nucleocapsid sequence, SARS-CoV-2 Spike sequence, SARS-CoV-2 ORF1ab sequence, SARS-CoV-2 matrix sequence, or SARS-CoV-2 envelope sequence. In some embodiments, the coronavirus SARS-CoV-2 target nucleic acid is specific to a SARS-CoV-2 variant. In some embodiments, the viral target nucleic acid is an HPV sequence. In some embodiments, the method can differentiate between two or more variants of SARS-CoV-2.

In some embodiments, the detecting occurs while the LAMP reactions are amplifying new products (e.g. real-time detecting of LAMP reaction products). In other embodiments, the detecting is after the LAMP reactions have ended or been halted (e.g. detecting LAMP reaction end products). In some embodiments, the LAMP reactions are halted by heating the LAMP reactions to a temperature of 80° C. or greater, for example, to inactivate the temperature resistant strand displacing DNA polymerase.

In some embodiments, the method of performing a plurality of LAMP reactions in parallel at a single temperature wherein at least one LAMP reaction comprises at least one LAMP-temperature shifting agent allowing for more efficient amplification at a set temperature compared to the same LAMP reaction in the absence of the at least one LAMP-temperature shifting agent comprises: (a) performing one or more LAMP reactions with a fluorescent intercalating-dye, (b) heating the one or more LAMP reactions at temperature of at least about 80 to 85° C., and (c) detecting a LAMP reaction product by detecting the fluorescent intercalating-dye. In some embodiments, the heating the one or more LAMP reaction is for at least 10-20 minutes at a temperature of at least about 80 to 85° C. In some embodiments, the fluorescent intercalating dye is selected from cyanine dye, acridine orange, ethidium bromide, methylene blue, propidium iodide, and propidium monoazide

Provided herein are methods for increasing end-point signal-to-noise ratios for a fluorescent intercalating-dye based LAMP assay by denaturing undesirable double stranded (ds) DNAs while preserving dsDNA pairing of desired LAMP reaction products using a heating step after the LAMP reaction(s) are completed. In some embodiments, the method comprises: (a) performing one or more LAMP reactions with a fluorescent intercalating-dye, (b) heating the one or more LAMP reactions at temperature of at least about 80, 85, 90, 95 or 100° C., and (c) detecting a LAMP reaction product by detecting the fluorescent intercalating-dye. In some embodiments, the heating the one or more LAMP reaction is for at least about 10, 20, 30, 40, 50 or 60 seconds at a temperature of at least about 80 to 85° C. to increase the signal to noise ratio in the detection step. In some embodiments, the fluorescent intercalating dye is selected from cyanine dye, acridine orange, ethidium bromide, methylene blue, propidium iodide, and propidium monoazide.

### Kits

Provided herein is a kit comprising reagents for performing a plurality of LAMP reactions in parallel at a set temperature wherein at least one LAMP reaction is not optimized for the set temperature in the absence of at least one LAMP-temperature shifting agent additive. In some embodiments, the kit comprises a composition described herein and an additional reagent or device. In some embodiments, the kit includes instructions for employing the kit components as well the use of any other reagent not included in the kit. Instructions may include variations that can be implemented. In some embodiments, the device comprises a plurality of cells for physically separating respectively a plurality of LAMP reactions.

In some embodiments, the kit comprises a LAMP-temperature shifting agent selected from: dimethyl sulfoxide (DMSO), ethanol, glycerol, glycogen, and guanidine hydrochloride (Gu-HCl).

In some embodiments the kit comprises a LAMP reaction product detection reagent and/or system. In some embodiments, the LAMP reaction product detection reagent is a detectable agent present in the primer set composition. In some embodiments, the LAMP reaction product detection system comprises a light source and a colorimetric sensor and/or light sensor. In some embodiments, the detectable agent includes, without limitation, a colorimetric agent, fluorophore, and/or luminescent agent (e.g., bioluminescent or chemiluminescent agent).

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

As used herein and in the appended claims, the singular forms “a”, “an” and “the” include plural reference, unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. In addition, the terms “comprising”, “including” and “having” can be used interchangeably.

As used herein, “about” means within 5% of a stated concentration range or within 5% of a stated time frame.

Various embodiments and aspects of the present invention as delineated hereinabove and as claimed in the claims section below find experimental support in the following examples which, together with the above descriptions, illustrate some embodiments of the invention in a non-limiting fashion.

### EXAMPLES

In the following Examples, methods are described for enhancing synergy between LAMP assays for detection of different nucleic acid sequences by altering the properties of interactions (e.g. melting energy input, assay temperature, and/or dsDNA bond strength) between a Bst DNA Polymerase with double-stranded DNA templates and LAMP primers in different LAMP reactions. Such methods are reliant upon the addition of an additive (referred to above as a LAMP-temperature shifting agent) with the primary effect of altering amplification product melting temperature so as to allow simultaneous amplification of different LAMP reaction products using a single, homogenous temperature setting.

### Example 1: Modulating LAMP Reaction Temperatures With Temperature-Shifting Additives to Optimize Conditions Away From the Ideal Reaction Temperature

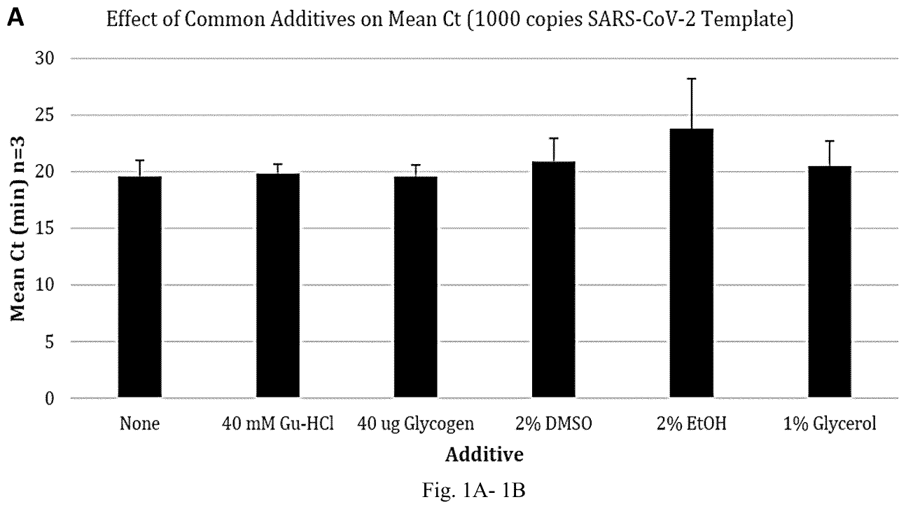

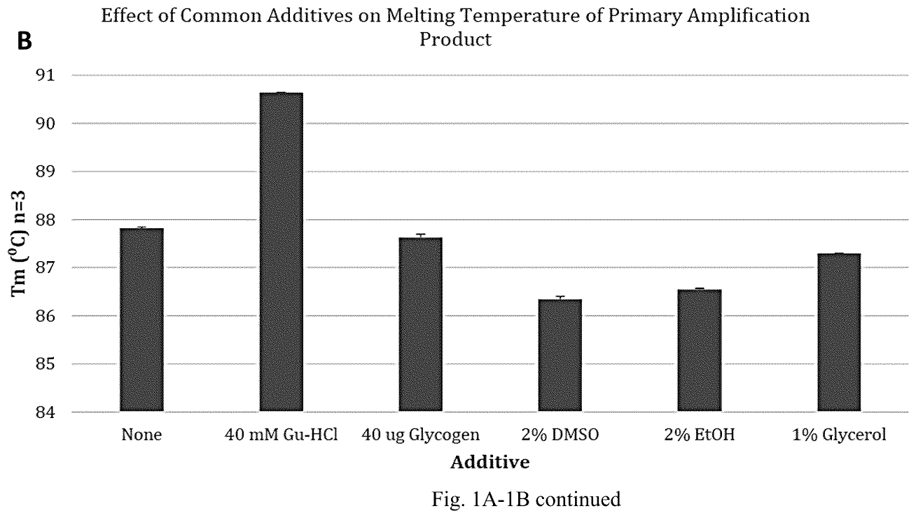

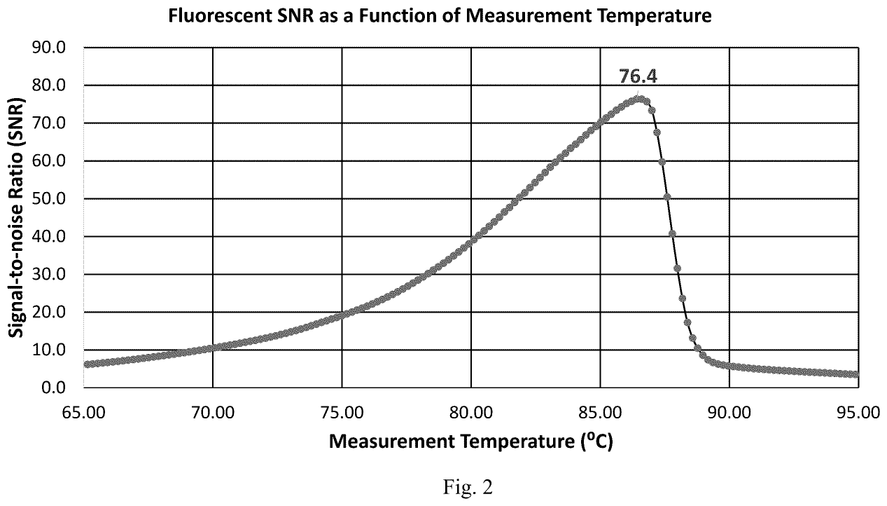

In order to run multiple LAMP assays on a single microfluidic chip with a single heating element, the spatial organization of the assay wells and design of the heating element must be such that each individual assay is within its optimal amplification temperature window. Due to the restrictions in primer design imposed by the use of LAMP technologies, it is not feasible to ensure identical, or even highly similar optimal heating ranges for each assay. Instead of implementing a multi-element heating solution, we propose the use of chemical additives capable of shifting the optimal temperature range of a given LAMP reaction by altering the intermolecular forces between primers, template, and Bst DNA polymerase. The additives that we have tested include: dimethyl sulfoxide (DMSO), ethanol, glycerol, glycogen, and/or guanidine hydrochloride (Gu-HCl), see FIG. 1. All experiments in this Example were carried out on 1000 copies of SARS-CoV-2 synthetic genomic RNA.

These data show that without substantial differences in amplification speed, the same template will melt at differential temperatures under the influence of different common additives such as guanidine-HCl, glycogen, DMSO, EtOH, or Glycerol at low concentrations.

Concurrent LAMP optimal temperature ranges can thus be modulated via the addition of these additives in different amounts depending on the unadulterated and ideal template melting temperatures. For instance, if targets A, B, and C, have optimal amplification temperatures at 62, 65, and 67° C., respectively, then addition of 40 mM Gu-HCl, to the assay well for detection of target A and 2% DMSO to the assay well for detection of target C will result in an even optimal amplification temperature for all assays.

The implementation of these common and cost-effective substances in this manner will allow each assay to be run in parallel from a single-temperature heating element. Although Gu-HCl (but not DMSO) is considered a health risk for an unskilled assay operator, our implementation includes the substances within the lyophilized reagent pellets sealed within the microfluidic device, and thus the user should never come into contact with the reagents.

### Example 2: Increasing End-Point Signal-To-Noise for Fluorescent Intercalating-Dye LAMP Assays

In another aspect, provided herein are methods for increasing end-point signal-to-noise ratios in fluorescent LAMP assays by specifically denaturing primer dimers and other non-specific amplification products while retaining the fluorescence of primary amplicons.

The detection of LAMP amplification products is dependent, in fluorescent implementations, upon the incorporation of a non-sequence specific dsDNA-intercalating dye, SYTO9. End-point measurements are, however, often hindered in their ability to demonstrate obviously significant signal-to-noise ratios due to background amplification of negative control samples. This background amplification has two likely causes: (1) non-specific amplification of template or contaminating nucleic acids, and (2) homo or hetero-dimer extension within the LAMP environment. Both of these sources of contaminating signal create highly truncated products with lengths less than that of the primary amplicon.

To separate the non-specific from specific intercalating dye signal we show that heating of the sample to a temperature above the melting temperature of the non-specific dimers and products, but below the melting temperature of the primary amplicon allows dissociation of the dye from only non-specific products while retaining the fluorescence of the real products. All experiments of this Example were carried out on 1000 copies of SARS-CoV-2 synthetic genomic RNA.

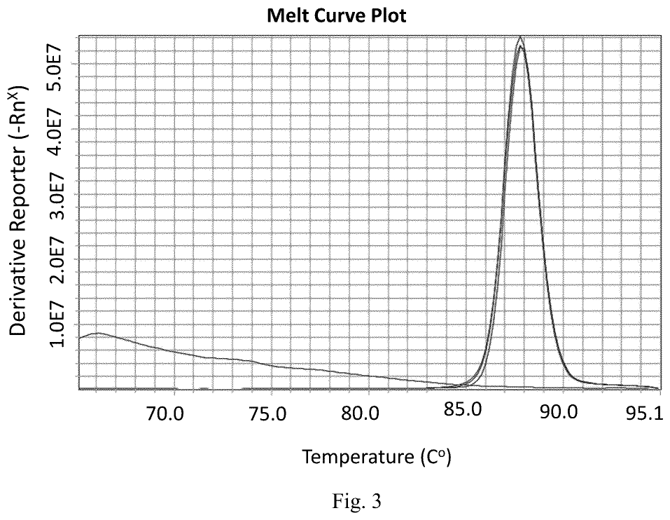

FIG. 3 shows SNR (defined as raw positive sample fluorescence/raw negative sample fluorescence for the same temperature point) in a range from 65° C. to 95° C. [n=3]. The reason for the distinctive shape is that up to about 85° C. all non-specific amplification products and primer dimers dissociate from the dye while the primary amplicon stays in-tact.

The following figure plots melting temperature against the change in intercalating dye fluorescence as the temperature is slowly increased at a rate of 0.05° C. per second.

The derivative reporter is a measure of how much intercalating dye is dissociating from the dsDNA substrate at a given temperature. The tailed curve peaking around 65° C. represents the loss of intercalating dye-non-specific dsDNA complexes up to around 85-90° C. The overlaid peaks at about 87.5° C. represent positive control samples with only a single species of proper dsDNA amplicon.

Heating the wells of the microfluidic chip after a standard amplification incubation period of 20-30 minutes, demonstrated an improvement in SNR (defined as positive control/negative control fluorescence) from about 2 to 76.4 in assays optimized for the detection of SARS-CoV-2 nucleocapsid gene.

These examples represent novel advancements to the field of LAMP-based nucleic acid diagnostics for pathogen detection including SARS-CoV-2 , high-risk human papilloma virus (HPVs), or any other pathogen with suitable amplification sites present in its RNA or DNA genome.

The present invention has been described in terms of one or more preferred embodiments, and it should be appreciated that many equivalents, alternatives, variations, and modifications, aside from those expressly stated, are possible and within the scope of the invention.

### EMBODIMENTS OF THE INVENTION

The following are embodiments of the invention:
1. A method of performing a plurality of loop-mediated isothermal amplification (LAMP) assays in parallel at a single temperature, the method comprising:
(a) providing a plurality of LAMP reactions, wherein each LAMP reaction comprises a LAMP primer set, a sample comprising nucleic acids, and a temperature-resistant strand displacing DNA polymerase and wherein at least one LAMP reaction comprises at least one LAMP-temperature shifting agent allowing for more efficient amplification at a set temperature compared to the same LAMP reaction in the absence of the at least one LAMP-temperature shifting agent;
(b) performing the plurality of LAMP reactions at the set temperature, wherein at least two of the LAMP reactions comprise different LAMP primer sets; and
(c) detecting LAMP reaction products produced by the plurality of LAMP reactions.
2. The method of embodiment 1, wherein each LAMP reaction comprises an equivalent sample.
3. The method of embodiment 1 or embodiment 2, wherein each LAMP reaction in step (a) further comprises deoxyribonucleotides and divalent metal ion suitable for DNA synthesis, and optionally wherein the plurality of LAMP reactions further comprises a reverse transcriptase.
4. The method of any one of embodiments 1-3, wherein the set temperature in step (b) is controlled by a water bath, heating element, and/or thermal cycler.
5. The method of any one of embodiments 1-4, wherein the plurality of LAMP reactions are in a plurality of cells and wherein the cells are optionally within a plate, linked test tubes or a microfluidic chip.
6. The method of any one of embodiments 1-5, wherein step (c) comprises detecting light absorption, light emission upon excitation, or light scattering before and after step (b); and optionally detecting a change in color, turbidity, or fluorescence after step (b) compared to before step (b).
7. The method of any one of embodiments 1-6, wherein the temperature-resistant strand displacing DNA polymerase is a B. stearothermophilus polymerase.
8. The method of any one of embodiments 1-7, wherein the LAMP-temperature shifting agent is selected from one or more of dimethyl sulfoxide (DMSO), ethanol, glycerol, glycogen, and guanidine hydrochloride (Gu-HCl).
9. The method of embodiment 8, wherein DMSO is present in the LAMP reaction between about 1 to 5% volume per volume (v/v).
10. The method of embodiment 8, wherein ethanol is present in the LAMP reaction between about 1.5 to 3% v/v.
11. The method of embodiment 8, wherein glycerol is present in the LAMP reaction between about 0.5 to 5% v/v.
12. The method of embodiment 8, wherein glycogen is present in the LAMP reaction between about 1 to 4 micrograms per microliter.
13. The method of embodiment 8, wherein Gu-HCl is present in the LAMP reaction between about 5 to 100 millimolar.
14. The method of any one of embodiments 1-13, wherein the sample is from a subject; and optionally wherein the sample from the subject is a nasopharyngeal swab or saliva sample.
15. The method of any one of embodiments 1-14, wherein the LAMP primer set is specific for SARS-CoV-2.
16. The method of embodiment 15, wherein the LAMP assay can differentiate between two or more variants of SARS-CoV-2.
17. The method of any one of embodiments 1-16, further comprising after step (b) and before step (c), a step (c′) comprising: (c′) heating the plurality of LAMP reactions to a temperature of at least about 80° C.
18. The method of embodiment 17, wherein step (c) comprises detecting a fluorescent intercalating-dye; and optionally wherein the fluorescent intercalating dye is selected from cyanine dye, acridine orange, ethidium bromide, methylene blue, propidium iodide, and propidium monoazide.
19. A method for increasing an end-point signal-to-noise ratio of a fluorescent intercalating-dye based LAMP assay, the method comprising:
(a) performing one or more LAMP reactions with a fluorescent intercalating-dye;
(b) heating the one or more LAMP reactions at temperature of at least about 80° C. for at least about 30 seconds; and
(c) detecting a LAMP reaction product by detecting the fluorescent intercalating-dye.
20. The method of embodiment 19, wherein the intercalating-dye is selected from: a cyanine dye, acridine orange, ethidium bromide, methylene blue, propidium iodide, and propidium monoazide.

We claim:

1. A method of performing a plurality of loop-mediated isothermal amplification (LAMP) assays in parallel at a single temperature, the method comprising:
(a) providing a plurality of LAMP reactions, wherein each LAMP reaction comprises a LAMP primer set, a sample comprising nucleic acids, and a temperature-resistant strand displacing DNA polymerase, and wherein at least one LAMP reaction comprises at least one LAMP-temperature shifting agent allowing for more efficient amplification at a set temperature compared to the same LAMP reaction in the absence of the at least one LAMP-temperature shifting agent:
(b) performing the plurality of LAMP reactions at the set temperature, wherein at least two of the LAMP reactions comprise different LAMP primer sets; and
(c) detecting LAMP reaction products produced by the plurality of LAMP reactions.

2. The method of claim 1, wherein each LAMP reaction comprises an equivalent sample.

3. The method of claim 1, wherein each LAMP reaction in step (a) further comprises deoxyribonucleotides and a divalent metal ion suitable for DNA synthesis, and optionally wherein the plurality of LAMP reactions further comprises a reverse transcriptase.

4. The method of claim 1, wherein the set temperature in step (b) is controlled by a water bath, heating element, and/or thermal cycler.

5. The method of claim 1, wherein the plurality of LAMP reactions are in a plurality of cells and wherein the cells are optionally within a plate, linked test tubes or a microfluidic chip.

6. The method of claim 1, wherein step (c) comprises detecting light absorption, light emission upon excitation, or light scattering before and after step (b); and optionally detecting a change in color, turbidity, or fluorescence after step (b) compared to before step (b).

7. The method of claim 1, wherein the temperature-resistant strand displacing DNA polymerase is a B. stearothermophilus polymerase.

8. The method of claim 1, wherein the LAMP-temperature shifting agent is one or more of dimethyl sulfoxide (DMSO), ethanol, glycerol, glycogen, and guanidine hydrochloride (Gu-HCl).

9. The method of claim 8, wherein DMSO is present in the LAMP reaction between about 1 to 5% volume per volume (v/v).

10. The method of claim 8, wherein ethanol is present in the LAMP reaction between about 1.5 to 3% v/v.

11. The method of claim 8, wherein glycerol is present in the LAMP reaction between about 0.5 to 5% v/v.

12. The method of claim 8, wherein glycogen is present in the LAMP reaction between about 1 to 4 micrograms per microliter.

13. The method of claim 8, wherein Gu-HCl is present in the LAMP reaction between about 5 to 100 millimolar.

14. The method of claim 1, wherein the sample is from a subject; and optionally wherein the sample from the subject is a nasopharyngeal swab or saliva sample.

15. The method of claim 1, wherein the LAMP primer set is specific for SARS-CoV-2.

16. The method of claim 15, wherein the LAMP assay can differentiate between two or more variants of SARS-CoV-2.

17. The method of claim 1, further comprising after step (b) and before step (c), a step (c′) comprising:
(c′) heating the plurality of LAMP reactions to a temperature of at least about 80° C.

18. The method of claim 17, wherein step (c) comprises detecting a fluorescent intercalating-dye; and optionally wherein the fluorescent intercalating dye is selected from cyanine dye, acridine orange, ethidium bromide, methylene blue, propidium iodide, and propidium monoazide.

19. A method for increasing an end-point signal-to-noise ratio of a fluorescent intercalating-dye based LAMP assay, the method comprising:
(a) performing one or more LAMP reactions with a fluorescent intercalating-dye;
(b) heating the one or more LAMP reactions at a temperature of at least about 80° C. for at least about 30 seconds; and
(c) detecting a LAMP reaction product by detecting the fluorescent intercalating-dye.

20. The method of claim 19, wherein the intercalating-dye is selected from: a cyanine dye, acridine orange, ethidium bromide, methylene blue, propidium iodide, and propidium monoazide.
